(19) United States
(12) Patent Application Publication
(10) Pub. No.: US 2026/0266807 A1
(43) Pub. Date: Sep. 10, 2026

(54) HIGH-BRIGHTNESS FLUORESCENT PROBES

(71) Applicant: Michigan Technological University, Houghton, MI (US)

(72) Inventors: Yoke Khin Yap, Houghton, MI (US); Xiuling Liu, Ann Arbor, MI (US); Nazmiye Yapici, South Lyon, MI (US); Dongyan Zhang, Houghton, MI (US)

(21) Appl. No.: 19/165,210

(22) PCT Filed: Apr. 10, 2023
(86) PCT No.: PCT/US2023/018026

§ 371 Date: Sep. 15, 2025

(51) Int. Cl. G01N 33/533; B82Y 15/00; G01N 33/68

(52) U.S. Cl. CPC G01N 33/533; B82Y 15/00; G01N 33/6854

(57) ABSTRACT

A compound includes a nanodot carrier, at least one radical-derived moiety covalently bond with the carrier, and at least one linker having first and second functional groups. The first functional group is linked to the at least one radical-derived moiety, and the second functional group is linked to at least one of a biomolecule, an oligonucleotide, and an aptamer. A method of making a probe is also disclosed.

### CROSS-REFERENCE TO RELATED APPLICATIONS

The application claims priority to International Application No. PCT/US2023/018026 filed Apr. 10, 2023. The disclosure of which is hereby incorporated by reference in its entirety.

### STATEMENT REGARDING FEDERALLY-SPONSORED RESEARCH

The inventions described herein were made with government support under Grant #2034693 awarded by the National Science Foundation. The Government has certain rights in this invention.

### BACKGROUND

Fluorescent probes are compounds with fluorescent properties that have biomedical applications. For example, fluorescent probes can be used as markers for specific staining of biomolecules and biotinylated biomolecules. More particularly, fluorescent probes can be used to stain ribonucleic acids (RNAs), deoxyribonucleic acids (DNAs), and proteins, in various analytical methods, such as fluidic sensors, fluorescent imaging and spectroscopy.

For the purpose of specific staining, fluorescent probes should be conjugated with biomolecules such as RNAs, single-stranded DNAs, oligonucleotides, aptamers, antibodies, or streptavidin. However, reliable tracking and quantification of the fluorophores are challenging due to commercial fluorescent probes' low brightness and photostability. Therefore, a need exists for improved fluorescent probes with high brightness and photostability.

### SUMMARY

A compound according to an exemplary embodiment of this disclosure, among other possible things includes a nanodot carrier, at least one radical-derived moiety covalently bond with the carrier, and at least one linker having first and second functional groups. The first functional group is linked to the at least one radical-derived moiety, and the second functional group is linked to at least one of a biomolecule, an oligonucleotide, and an aptamer.

In a further example of the foregoing, the compound includes a second linker having first and second functional groups and a second radical-derived moiety. The first functional group of the second linker is linked to the second radical-derived moiety, and the second functional of the second linker group is linked to a fluorescent entity.

In a further example of any of the foregoing, the nanodot carrier has at least one polar group. The first and second radical-derived moieties are each covalently bonded with the carrier at one of the at least one polar groups.

In a further example of any of the foregoing, the at least one polar group is a hydroxyl (—OH) group.

In a further example of any of the foregoing, the nanodot carrier is an h-BN nanodot carrier.

In a further example of any of the foregoing, wherein the nanodot carrier has dimensions between about 1 and about 100 nm.

In a further example of any of the foregoing, the nanodot carrier has dimensions between about 1 and about 20 nm.

In a further example of any of the foregoing, the nanodot carrier comprises less than 30 layers of h-BN.

In a further example of any of the foregoing, the nanodot carrier comprises between about 1 and 10 layers of h-BN.

In a further example of any of the foregoing, at least ten radical-derived moieties are covalently bonded with the carrier. Each radical-derived moieties is linked to a linker. Each linker is linked to at least one of a moiety, a biomolecule, an aptamer, or an oligonucleotide.

In a further example of any of the foregoing, the moiety is a chelating agent.

A method of making a probe according to an exemplary embodiment of this disclosure, among other possible things includes mechanically processing nanodots in polar liquid to create imperfections on the nanodots, treating the nanodots to provide polar groups at the imperfections, associating radicals with the polar groups at the imperfections, and linking a linker with the nanodots via one of the associated radicals at a first functional group of the linker.

In a further example of the foregoing, the method includes linking a fluorescent entity to a second functional group of the linker.

In a further example of any of the foregoing, the mechanically processing includes agitation.

In a further example of any of the foregoing, the agitation is accomplished by sonication or by homogenizer.

In a further example of any of the foregoing, the treating is an acid treatment. The polar groups are hydroxyl (—OH) groups.

In a further example of any of the foregoing, the method also includes using the probe for fluorescence in-situ hybridization (FISH) to detect or quantify at least one of RNAs, DNAs, genes, and proteins via oligonucleotide-conjugated antibodies in fixed or live cell samples or fixed or live tissue samples.

In a further example of any of the foregoing, the probe is configured to be hybridized on the oligonucleotide-conjugated antibodies.

In a further example of any of the foregoing, the method also includes using the probe in spatial omics to image, localize or map at least one of RNAs, DNAs, genes, and proteins via oligonucleotide-conjugated antibodies in fixed or live cell samples or fixed or live tissue samples.

In a further example of any of the foregoing, the probe is configured to be hybridized on the oligonucleotide-conjugated antibodies.

In a further example of any of the foregoing, the linker is a first linker. The method also includes linking a second linker with the nanodots via a radical-derived moieties of the associated radicals at a first functional group of the second linker, linking a fluorescent entity to a second functional group of the first linker, and linking an oligonucleotide, an apatamer, or a biomolecule to a second functional group of the second linker.

In a further example of any of the foregoing, an oligonucleotide is linked to the second functional group of the second linker. The method also includes using the probe in a sensor to detect proteins or biomarkers extracted from body fluids. The oligonucleotide is configured to bind with the proteins or biomarkers.

In a further example of any of the foregoing, the biomolecule is streptavidin. The method also includes using the probe to detect biotinylated molecules.

In a further example of any of the foregoing, the biomolecule is an antibody. The method also includes using the probe to detect proteins inside a cell, outside cells, or on a cell surface.

In a further example of any of the foregoing, the method also includes using the probe to detect proteins on extracellular vesicles (EV) and exosomes.

### BRIEF DESCRIPTION OF THE DRAWINGS

FIG. 1A schematically shows an example high-brightness fluorescent probe (HBP).

FIG. 1B schematically shows the synthesis of an example HBP like the HBP of FIG. 1A from a boron nitride (BN) dot carrier.

FIG. 2 shows TEM (transmission electron microscopy) images of BN dots.

FIG. 3 compares the absorbance (abs) and fluorescence (emission, Em) spectra of an example fluorescent entity and BN dots conjugated with the example fluorescent entity.

FIGS. 4A-D show the absorbance (Abs) and fluorescence (emission, Em) spectra of BN dots conjugated with Sulforhodamine alkyne, AlexaFluor™ alkyne (AF568 and AF555), and Atto alkyne (Atto 488), respectively.

FIG. 5 shows the changes in fluorescence intensity as a function of UV lamp irradiation time for free fluorescent entity (FAM dye) and BN dots conjugated with a fluorescent entity.

FIG. 6A schematically shows hybridization of BN dots conjugated with an example fluorescent entity and oligonucleotides on biotin-functionalized oligonucleotides with complementary sequence (CS), and capturing on the streptavidin-coated beads.

FIG. 6B schematically shows mixing of BN dots conjugated with an example fluorescent entity and oligonucleotides, on biotin-functionalized oligonucleotides with non-complimentary sequence (NCS), and capturing of the biotinylated NCS on the streptavidin-coated beads.

FIG. 6C compares the fluorescence images of micro-beads of FIGS. 6A-B compared with the cases with commercial counterparts.

FIG. 7A schematically shows the hybridization of the target RNAs with primary probes and secondary probes.

FIG. 7B shows microscopic fluorescence images of the beta-actin RNAs inside HeLa cells stained with probes (upper panel) and commercial probes (lower panel).

FIG. 8A schematically shows staining of a targeted protein with an oligonucleotide-conjugated antibody.

FIG. 8B schematically shows the subsequent hybridization of the HBP on the targeted protein of FIG. 8A.

FIGS. 9A-B shows the gel image staining and fluorescent spectra of elutions from the experiment of FIG. 9A for an HBP with another example fluorescent entity and an example protein.

FIGS. 10A-B shows the gel image staining and fluorescent spectra of elutions from the experiment of FIG. 10A for an HBP with another example fluorescent entity and an example antibody.

FIG. 11A shows fluorescence signals from various fractions (E3 to E11) containing sample HBPs.

FIG. 11B shows UV-Vis spectra from fractions E3-E6 of those fractions in FIG. 11A.

FIG. 11C shows fluorescence spectra from fractions E3-E6 of those fractions in FIG. 11A.

### DETAILED DESCRIPTION

Generally, high-brightness fluorescent probes include a carrier element, radical-derived moieties covalently functionalized on the carrier element, linkers linking the carrier element via the radical-derived moieties with fluorescent moieties, and linkers linking the carrier element via the radical-derived moieties with biomolecules, oligonucleotides, or aptamers. For biomedical applications, each carrier element, linker, and fluorescent moiety must be biocompatible (though the requirements for biocompatibility will vary with the particular application).

One example carrier element is a nanomaterial, such as boron nitride nanoparticles (BN dots), boron nitride nanotubes (BNNTs), hexagonal boron nitride (h-BN) nanosheets, carbon nanoparticles, carbon nanotubes (CNTs), graphene nanosheets, aluminum nitride nanoparticles, silicon nitride nanoparticles, aluminum oxide nanoparticles, silicon nitride nanoparticles, transition metal dichalcogenide nanoparticles (TMDC such as MoS₂, WS₂, MoSe₂, WSe₂, MoTe₂), peptides, or polymers. However, it has been shown that fluorescent elements linked to CNTs exhibit quenching, or a reduction in the quantum yield and brightness of the fluorescence.

It has been discovered that certain fluorescent probes having nanomaterial carriers not only do not exhibit the quenching effect but also exhibit brightness one or more orders of magnitude higher than other known fluorescent probes, as will be discussed herein.

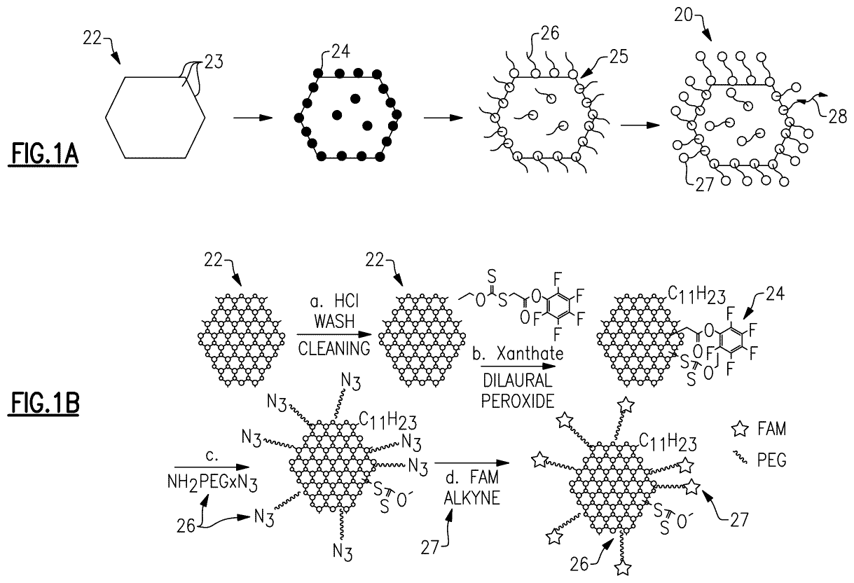

Referring now to FIG. 1A, a sequence of steps for making a high-brightness fluorescent probe (HBP) 20 is shown. A nano-scale “nanomaterial” carrier 22 is provided with imperfections 23 at the edges and/or surface. The nanomaterial carrier 22 is dispered in an organic liquid such as chloroform and dichloroethane. Radicals generated in the reaction liquid covalently bond with the imperfections 23 to form precursor molecules 24. Linkers 26 are then reacted with precursor molecules 24, dissociate the precursor molecules 24 into leaving groups (free by-product moieties) and retain the radical-derived moieties 25 on the carrier 22. The radical-derived moieties 25 are now connected to a first end of the linkers 26. A second, opposite end of each linker 26 links the carrier 22 to at least one biomarker, oligonucleotide, or aptamer 28 or to at least one moiety 27. The resulting HBP 20 has a carrier 22 bearing at least one biomarker/oligonucleotide/aptamer 28 and at least one moiety 27 via the radical-derived moieties 25 and linkers 26.

Carrier 22 is, in one example, a processed BNNT. In the example of FIG. 1A, carrier 22 is a zero-dimensional BN “dot” (e.g., the size of the dot in all three dimensions is on the nano-scale, or less than about 100 nm), though other carriers could also be used. In a more particular example, all three dimensions of a dot carrier are less than about 20 nm. Other example carriers 22 are multi-walled BNNTs or CNTs, where each BNNT or CNT has multiple co-axial shells of hexagonal boron nitride (h-BN for BNNTs) or graphene (for CNTs), with a typical external diameter of more than about 0.4 nm but less than about 100 nm. The length of these BNNTs and CNTs is between about 1-100 nm. In other examples, carrier 22 can be another nano-scale particles, such as hexagonal boron nitride nanosheets/nanoparticles, graphene/graphite nanosheets/nanoparticles, any transition metal dichalcogenide (TMDCs) nanosheets/nanoparticles, any nanosheets/nanoparticles of layered materials (materials with covalent layered structures that bond with van der Waals forces between layers), aluminum nitride nanoparticles, silicon nitride nanoparticles, aluminum oxide nanoparticles, silicon nitride nanoparticles, peptides, or polymers.

FIG. 1B illustrates an example process for functionalizing carriers 22 so that the carrier 22 is bonded with the precursor molecules 24. It should be understood that the particular elements shown in FIG. 1B are exemplary only, and other elements could be used in their place, as will be discussed in detail below. As illustrated in FIG. 1B, carrier 22 is briefly washed, for example, with HCl to remove potential contaminants in an optional step (step a). After removing the washing medium, carrier 22 is reacted with, for example, but not limited to, peroxides and xanthates such that free radicals (step b) will bond on the carrier 22 to form various precursor molecules 24 (outcome of step b). The reaction materials may be added to the reaction liquid in which the carrier 22 is dispersed. Similar kinds of radical functionalization, by decomposition of diazonium salt and peroxides, are known for the functionalization of CNTs. However, it was previously thought that these kinds of radical functionalization could not functionalize BNNTs and h-BN nanosheets due to their chemically inert nature, which is discussed in more detail below.

Typical methods to radically functionalize BNNTs and h-BN nanosheets require particular chemical treatment. For example, but not limited to, prolonged (more than five hours) auto-clave or heating process in peroxide, hydrazine, HNO₃, H₂SO₄ and oleum at high temperatures (~75-160° C.), prolonged treatment (more than five hours at 100° C.) in stearoyl chloride, or treatment by isophorone Diisocyanate (IPDI) may be required.

It has been discovered that BNNTs and BN dots can be functionalized with radicals as discussed above without prolonged chemical treatments in auto-clave at high temperatures by pretreating the BNNTs or BN dots with mechanical processing in solution or solvent, to be described hereafter.

The example linker 26 shown in FIG. 1B, NH₂PEGₓN₃, (x between 1 to about 100) has two or more functional groups, R and R′. The linker 26 can be other known polymers. The functional groups R and R′ are reactive groups that facilitate the covalent bonding of the linker 26 to other structures by any known chemistry. R and R′ can be the same or different functional groups. For example, R and R′ can be amine and azide, respectively. R and R′ can be any known functional groups such as ethoxsilane groups, carboxylic acid, isothiocyanate, maleimide, an alkyne group, a hydroxyl group, a thiol group, monosulfone, or an ester group such as a succinimidyl, sulfodichlorophenol, pentafluorophenyl or tetrafluorophenyl. The linker 26 can be any type of molecule with two or more functional groups, R and R′. One example of linker 26 is a linear or branched polymeric molecule. In some examples, the linker 26 has a length of less than about 200 nm. In some examples, multiple linkers 26 can be connected in series.

The R functional group (amide in the example of FIG. 1B, step c) will covalently bond to precursor molecules 24 functionalized on the carrier 22 according to known chemistry. Such a reaction will release free leaving groups (not shown in the figure), and retain a portion of the precursor molecules 24 on the carrier 22 as the radical-derived moieties 25 shown in FIG. 1A. This process allows the linker 26 to covalently bond to the carrier 22 via the radical-derived moieties 25. The functional group R′ (azide, N₃ in the example of FIG. 1B, step c) can covalently connect with multiple fluorescent moieties 27 (for example, Fluorescein dye FAM in FIG. 1B, step d) by click reaction with alkyne functionalized-FAM. The functional group R′ can covalently bind with biomolecules, oligonucleotides or aptamers 28 by a similar click reaction. Moities 27 and biomolecules, oligonucleotides or aptamers 28 can be connected to the functional group R′ simultaneously or one after another.

The moiety 27 is, in one example, a fluorescent (FAM) entity. The fluorescent entity could be any fluorescent dye known in the art, including but not limited to coumarins, benzoxadiazoles, acridones, acridines, bisbenzimides, indole, benzoisoquinoline, naphthalene, anthracene, xanthene, pyrene, porphyrin, fluorescein, rhodamine, boron-dipyrromethene (BODIPY) and cyanine derivatives. Many such fluorescent dyes are commercially available. The fluorescent entity can also include tandem dyes with two different dyes connected and interacting via FRET (fluorescence resonance energy transfer). The fluorescent entity covalently interacts with the functional group R′ of linker 26 as discussed above.

In other examples, moiety 27 is a labeling moiety or other moieties to be delivered to a biological system (living body, cells, biological sample, etc.) by the carrier 22, such as antibodies, peptides, DNAs, RNAs, oligonucleotides, or the like.

The moiety 27, in other examples, can be molecules and chelating agents with radioactive isotopes, ferromagnetic, magnetic elements, and/or other elements, for example and not limited to rare-earth elements (Lanthanum, Cerium, Praseodymium, . . . ), elements of the Lanthanide series, ionic oxides, etc. Non-biological molecules could also be used. In these examples, HBP 20 (in FIG. 1A) can be used as a contrast agent for medical imaging such as positron emission tomography (PET), single-photon emission computerized tomography (SPECT), computerized tomography (CT), magnetic resonance imaging (MRI), etc.

In another example, moiety 27 can include combinations of any of the examples discussed above. In this example, HBP 20 can be used as a heterogeneous probe for biomedical detection and sensing.

Some nanomaterial carriers 22, and in particular, boron nitride (BN)-based nanomaterials, are known to be chemically inert. Therefore, it has been difficult to functionalize prior art nanomaterial carriers for covalent interactions with other structures. However, it has been discovered that carriers 22, such as the BN dot carrier shown in FIG. 1A or BNNTs that have been subject to mechanical processing in solution or solvent, such as agitation, exhibit increased propensity to covalently interact with precursor molecules 24 without prolonged, pressurized, high-temperature chemical treatments. The solution/solvent can be the same solution/solvent in which source material is treated to form nanodots as discussed in more detail below, or a different solution/solvent. Furthermore, it has been discovered that the mechanical processing of nanomaterial carriers improves the solubility of the nanomaterial carriers in aqueous solutions, which can improve biocompatibility. Additionally, mechanical processing cuts carrier material into smaller pieces which can be desirable when forming dots, for example. Agitation can be accomplished by homogenizer and/or sonication, such as tip sonication or bath sonication, for instance.

Referring again to FIG. 1A, mechanical processing results in carrier 22 with imperfections 23. During mechanical processing in solution/solvent, imperfections 23 form on the carrier 22 such that localized polarities or charges are formed at the imperfections 23. Polar or charged groups from the solution/solvent interact with the localized polarities or charges at the imperfections. For the example in FIG. 1B, the carrier is an h-BN nanodot carrier 22. In this particular example, imperfections 23 are disruptions in the hexagonal structure of the boron nitride material, which disruptions have localized polarity imbalances. For example, for certain solvents/solutions, hydroxyl groups from the solvent/solution may interact with the imperfections 23, though other solvents/solutions may have other polar or charged groups that can interact with the localized imperfections 23, such as amino, carboxylic acids, or aldehyde groups, depending on the processing and type of solvent/solution.

In one method of making carriers 22, h-BN powder is treated with liquid nitrogen followed by sonication in room-temperature water/ethanol solution. In another method of making carriers 22, the h-BN powder is treated in dimethylformamide (DMF) or another polar solution/solvent for two to four hours by using a homogenizer. In one example, the treatment in a polar solvent is solvothermal (e.g., the solvent/solution is heated).

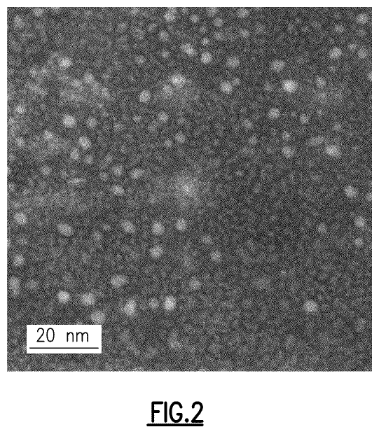

After the solvothermal treatment, the carrier 22 suspensions are centrifuged to precipitate large particles. In a particular example, the suspension is centrifuged at 10,000 rpm for 10 minutes. In this example, the size of the carriers 22 in the suspension is about ~3-5 nm after heat treatment and centrifugation, as confirmed by TEM (transmission electron microscopy) imaging shown in FIG. 2.

It has been discovered that making carriers 22 according to the above-described method leads to a production yield orders of magnitude higher than prior art methods. For example, for the method performed with 20-30 minutes of bath sonication, heat treatment for 7 to 12 hours while stirring with a magnetic stir bar, and centrifugation at 10,000 rpm for 10 minutes, the production yield is about 47%, as compared to the reported 1-26% for prior methods. Production yield is the weight percentage of h-BN bulk powder that become carriers 22 after the evaporation step discussed above.

For the example method using DMF solution, hydrocarbon groups or fragments from the solution interact with the localized polarities at the imperfections 23 of carriers 22, though other solutions may have other polar groups that can interact with the localized polarities, such as amino, carboxylic acids, aldehyde, etc. The carriers 22 can then undergo acid treatment according to any known method, which replaces the hydrocarbon groups or fragments with hydroxyl groups (—OH groups) at the imperfections 23 of carrier 22, which result in processed carriers (discussed in more detail below). Acid treatment also removes other contamination from the carriers 22, such as the hydrocarbon fragments of DMF. The processed carriers can then be linked to precursor molecules 24 by any known chemistry such as the example process of FIG. 1B to form radical-functionalized carriers.

The radical-functionalized carriers have increased capacity for attaching to linkers 26 and thus moieties 27, and biomolecules, oligonucleotides or aptamers 28, due to the reactive precursor molecules 24 compared to processed carriers 22. More specifically, the precursor molecules 24 act as reactive sites for covalently linking the processed carrier to linker 26 via functional group R. Accordingly, the brightness of the fluorescent probes 20 having a radical-functionalized carrier and fluorescent moieties/entities 27 is higher than prior art fluorophores because the radical-functionalized carrier can be linked to multiple fluorescent entities 27. More generally, the radical-functionalized carriers can be linked to more moieties 27 than processed carriers 22.

In a particular example, the BN dot carriers 22 that are processed to form processed carriers, as discussed above, have 4 layers of h-BN that are each about ~3-6 nm in diameter shown in FIG. 2. Each layer can bond to 10 or more linkers 26 and fluorescent entities 27 or other biomolecules, oligonucleotides or aptamers 28 after processing, as discussed above. Thus, the example processed carriers 22 can bond to 20 or more radicals 24, linkers 26, fluorescent entities 27, and biomolecules, oligonucleotides or aptamers 28, to form a high-brightness fluorescent probe 20. The high-brightness fluorescent probe 20 is thus 20 or more times brighter than a carrier with a single fluorescent entity. For branched linkers (n branches), the intensity will be as larger as 20 times that of a carrier with a single fluorescent entity.

In other examples, linker 26 is an amino-silane linkers. Other linkers 26 might have a variety of functional groups such as amino, carboxylic acid, succinimdyl ester, maleimide, carboimide, pyridyldithiol, haloacetyl, aryl azide, azide, alkyne, DBCO derivatives hydrazide and monosulfone groups. Those groups could be used for the conjugation of carriers 22 to dye, drug, or any targeting material. Cross-linkers which contain dual functional group can also be used to obtain functional group to conjugate linkers 26 to other entities such as dye, peptide, oligonucleotide, DNA, RNA, antibody, proteins, drugs or other nanoparticles. Those cross-linkers might be SMCC (succinimidyl 4-(N-maleimidomethyl)cyclohexane-1-carboxylate), sulfo-SMCC ((sulfo-succinimidyl 4-(N-maleimidomethyl)cyclohexane-1-carboxylate), AMAS (N-α-maleimidoacet-oxysuccinimide ester), BMPS (N-β-maleimidopropyl-oxysuccinimide ester), GMBS (N-γ-maleimidobutyryl-oxysuccinimide ester), sulfo-GMBS, MBS (m-maleimidobenzoyl-N-hydroxysuccinimide ester), sulfo-MBS, EMCS (N-ε-malemidocaproyl-oxysuccinimide ester), sulfo-EMCS, SMPB (succinimidyl 4-(p-maleimidophenyl)butyrate), sulfo-SMPB, SMPH (Succinimidyl 6-((beta-maleimidopropionamido)hexanoate), LC-SMCC succinimidyl 4-(N-maleimidomethyl)cyclohexane-1-carboxy-(6-amidocaproate), sulfo-KMUS (N-κ-maleimidoundecanoyl-oxysulfosuccinimide ester), SM(PEG)n where n=2, 4, 6, 8, 12, 24 (PEGylated SMCC cross-linker), SPDP (succinimidyl 3-(2-pyridyldithio)propionate), LC-SPDP, sulfo-LC-SPDP, SMPT (4-succinimidyloxycarbonyl-alpha-methyl-α(2-pyridyldithio)toluene), PEGn-SPDP (where n=2, 4, 12, 24), SIA (succinimidyl iodoacetate), SBAP (succinimidyl 3-(bromoacetamido)propionate), SIAP (succinimidyl (4-iodoacetyl)aminobenzoate), sulfo-SIAP, ANB-NOS (N-5-azido-2-nitrobenzoyloxysuccinimide), sulfo-SANPAH (sulfosuccinimidyl 6-(4′-azido-2′-nitrophenylamino)hexanoate), SDA (succinimidyl 4,4′-azipentanoate), sulfo-SDA, LC-SDA, sulfo-LC-SDA, SDAD (succinimidyl 2-((4,4′-azipentanamido)ethyl)-1,3′-dithiopropionate), Sulfo-SDAD, DCC (N, N′-Dicyclohexylcarbodiimide), 1-Ethyl-3-(3-dimethylaminopropyl) carbodiimide (EDC, EDAC or EDCI), EMCH (N-ε-maleimidocaproic acid hydrazide), MPBH (4-(4-N-maleimidophenyl)butyric acid hydrazide), KMUH (N-κ-maleimidoundecanoic acid hydrazide), PDPH (3-(2-pyridyldithio)propionyl hydrazide), PMPI (p-maleimidophenyl isocyanate), SPB (succinimidyl-[4-(psoralen-8-yloxy)]-butyrate), or other known linkers.

### Examples of High-Brightness Probes With Oligonucleotides 28 for Fluorescence In-Situ Hybridization (FISH)

In the example of FIG. 1A, moiety 27 is a fluorescent entity, and in particular, is FAM, which is a green dye. FAM can be conjugated to the linker 26 at R′ by any known chemistry. For instance, for the azide-amine linker 26 of FIG. 1B, a click reaction can be performed to covalently bond the R′ group of linker 26 to an alkyne group of FAM.

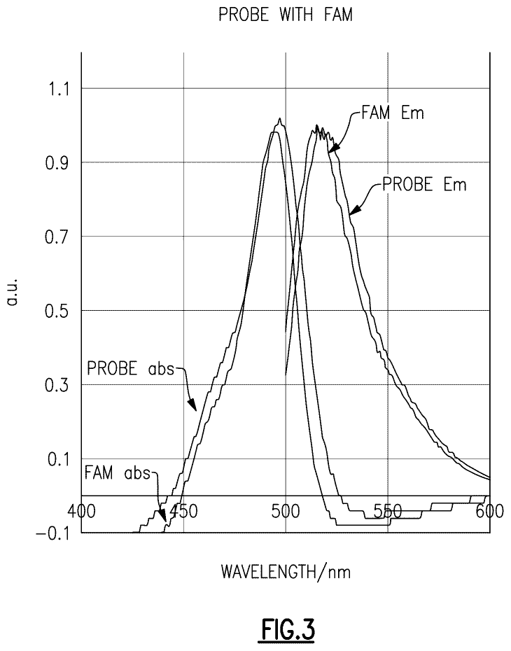

FIG. 3 shows the normalized absorption (abs) and emission (Em, fluorescent) spectra of FAM alkyne and example processed carrier 22 conjugated with moiety 27. The absorption (abs) peak and emission (Em) peak of FAM alkyne are ~494 nm and ~515 nm. The abs peak and Em peak are ~497 nm and ~518 nm, respectively, after purification by a size exclusion column (SEC), confirming the conjugation of the processed carrier 22 with precursor molecules 24, linkers 26, moieties 27 (FAM alkyne). The molar extinction coefficient of the processed carrier 22 conjugated with moiety 27 was determined as 2.37×10⁶ to 7.2×10⁶ L mol⁻¹cm⁻¹, higher than that of FAM dye (8.30×10⁴ L mol⁻¹cm⁻¹) by 28.6 to 86.7-time.

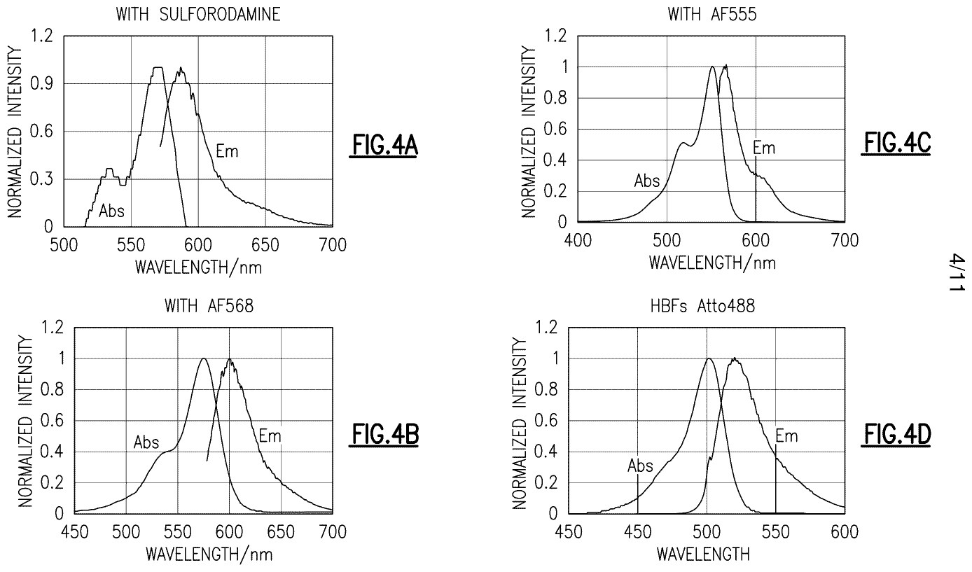

The same chemistry (e.g., click reaction discussed above) or other known chemistries can be applied to conjugate various fluorescent moiety 27 containing alkyne functional groups such as sulforhodamine alkyne, and sulfo-cy5.5 alkyne, to the processed carrier 22 via linkers 26. For additional examples, the carrier 22 can be conjugated to sulforhodamine alkyne, AlexaFluor™ alkyne (AF568 and AF555) (ThermoFisher Scientific Inc.), and Atto alkyne (Atto 488) (ATTO-TEC GmbH) via linker 26, as shown by the absorption and fluorescence spectra in FIGS. 4A-D.

Other moieties 27, such as alkyne-polyethylene glycol, alkyne antibodies, etc. can also be conjugated to the processed carrier 22 via linkers 26 using the same chemistry or other known chemistries. For example, alkyl antibodies can be made by reducing an antibody using DTT (Dithiothreitol), which results in reduced sulfuhydryl groups, which can then be connected to maleimide-PEG4-alkyne or another alkyne-containing moiety according to the known procedure. Other small molecules such as sugars, nitroxides, biotin, drugs, etc. or macromolecules, peptides, DNA, RNA sequences, and proteins such as SA (streptavidin and its derivatives) can also be covalently connected to the functionalized BN carrier 22/linker 26 according to known methods.

Though the preceding description of processed carrier 22 is made with respect to h-BN dots, carbon dots, and other nanoparticles (aluminum nitride, silicon nitride, aluminum oxide, TMDCs, etc. as discussed above) can be linked to linkers 26 by chemical means, such as by mechanical agitation discussed above, and then linked to moieties 27, as discussed above.

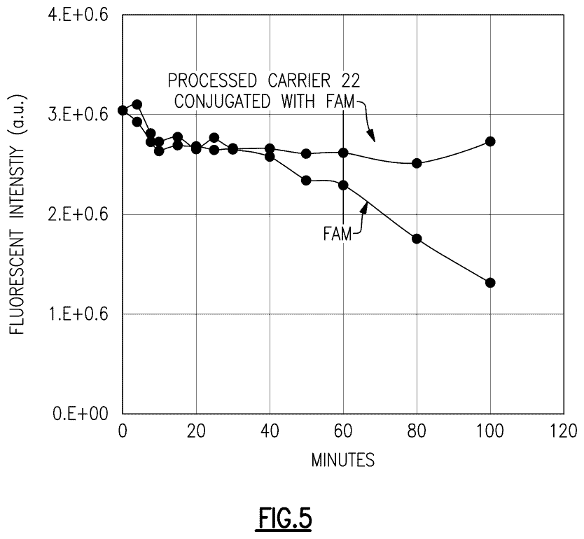

FIG. 5 shows the changes in fluorescent intensity of processed carrier 22 conjugated with moiety 27 (FAM Alkyne). After 100 min of irradiation of a Halogen lamp (250 W, 4 inches away from the samples), the fluorescent intensity of the FAM conjugated processed carrier 22 remained at 90% of the initial intensity. In contrast, the fluorescent intensity of free-standing moiety 27 (without being conjugated on carrier 22) dropped to 43% of the initial intensity. The high-brightness probes 20 with FAM alkyne offer >2×photostability than free-standing FAM alkyne.

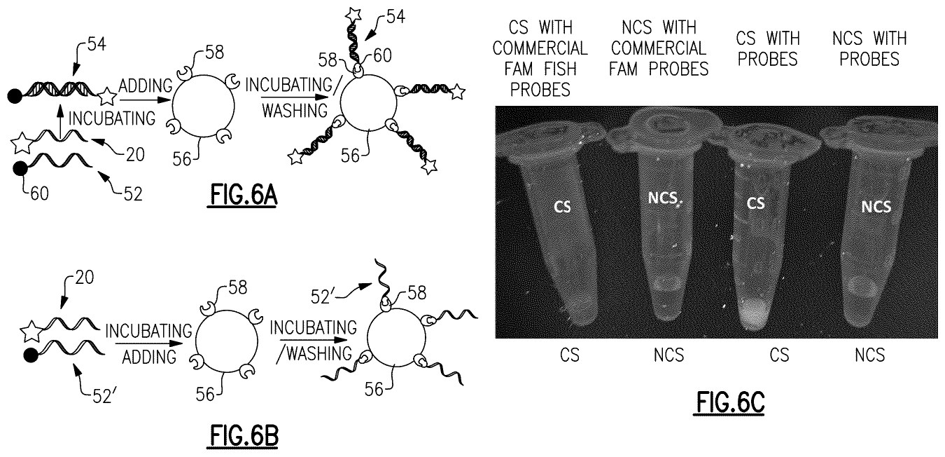

In one example, processed carrier 22 conjugated with moiety 27 (FAM alkyne) and oligonucleotides 28 can be used as HBPs 20 for fluorescence in-situ hybridization (FISH) to stain protein streptavidin specifically. FIG. 6A (top panel) illustrates example HBPs 20 hybridized with biotin-functionalized oligonucleotides with complementary sequence (CS) 52 to form hybrids 54. The hybrids 54 can then link with microbeads 56 coated with a protein 58 such as streptavidin via the biotin 60 and produce green fluorescence. For comparison purposes, the same hybridization was repeated with commercial FAM probes with one FAM dye per probe.

FIG. 6B shows a control where example HBPs 20 and commercial FAM probes were hybridized with biotin-functionalized oligonucleotides with non-complementary sequence (NCS) 52′. Since these are NCS, the example HBPs 20 and FAM probes will not be hybridized on the biotin-functionalized oligonucleotides 52′. In these cases, the biotin-functionalized oligonucleotides 52′ can bind with the streptavidin-coated micro-beads 56 but carry no dye and produce no fluorescence.

FIG. 6C shows the images of micro-beads collected in all cases discussed above under the irradiation of a UV lamp. As expected, no fluorescence can be observed in all the NCS cases. The CS case stained with commercial FAM probes shows very weak fluorescence. The CS case stained with example probes 20 shows strong fluorescence signifying the nature of high brightness.

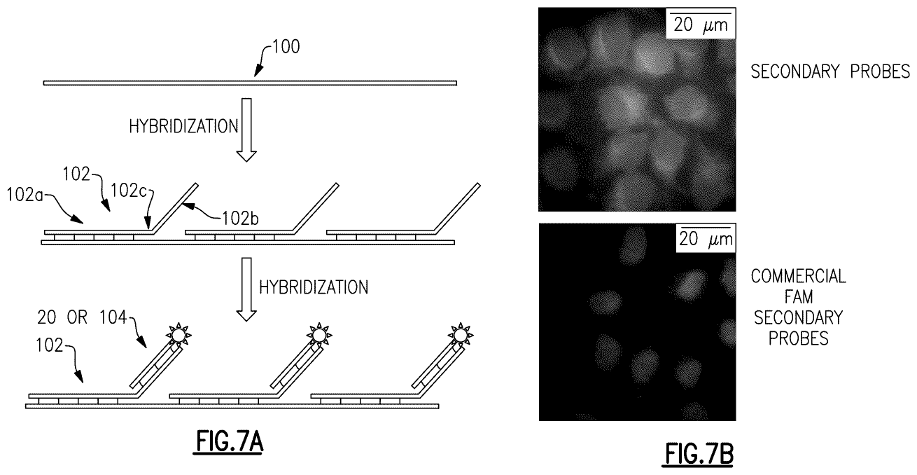

In one example, HBPs 20 conjugated with moiety 27 (FAM Alkyne) and oligonucleotides 28 can be used for fluorescence in-situ hybridization (FISH) to stain, detect, and localize messager-RNAs (beta-actin mRNAs) in fixed HeLa cells (in this case the probes act as secondary probes). FIG. 7A illustrates target RNA 100 hybridized with a series of oligonucleotides (primary probes 102) with complementary nucleotides (nt) at one end 102a. In one example, there are about 35 nt at the end 102a which interact with the target RNA 100. All these primary probes 102 have a bridge tail 102b at the opposite end. Example secondary HBPs 20, which can be the example HBPs 20 discussed herein, are then hybridized on the primary probes' 102 bridge tails 102b to complete the targeted RNAs' 100 staining. In one example, the bridge tail 102b and the HBPs 20 are about 20 nt long with complementary sequences. Between primary probe end 102a and bridge tails 102b, there is a short spacer oligonucleotide 102c, which is about four T bases.

FIG. 7B shows the fluorescence microscopy images of the fixed HeLa cells after hybridizing the primary probes 102 and secondary HBPs 20 using the example HBPs 20 described above. Example secondary HBPs 20 stain the mRNAs 100 inside the cytoplasm surrounding the nucleus. Bright green fluorescence was recorded from the example secondary HBPs 20 even without using the electron multiplier of the microscope. In contrast, the sub-image underneath shows no fluorescence signal from the fixed HeLa cells after hybridizing with the primary 102 and commercial FAM secondary probes. The fluorescence of commercial FAM secondary probes is too dim to be detected without using the electron multiplier of the microscope. This contrasting result shows the nature of the high brightness of HBP 20.

The use of the primary probe 102 in combination with the secondary HBP 20 allows for the use of secondary HBPs 20 that are agnostic to the target RNA 100. That is, each secondary HBP 20 corresponds to the bridge tail 102b of the primary probe 102, which can be selected as one of several optional bridge tail 102b nt sequences. On the other hand, the primary HBP 102 is selected to be specific to the target RNA 100.

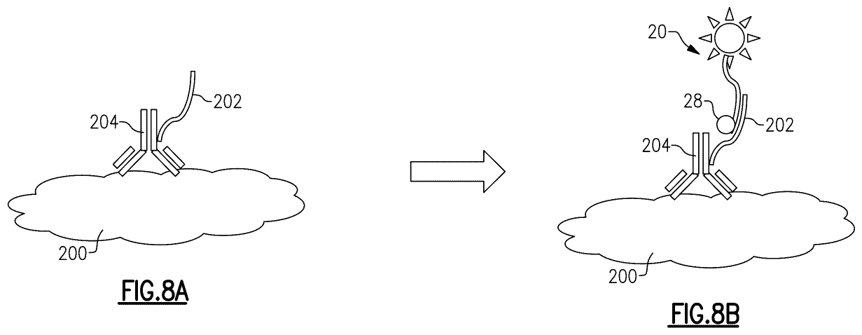

HBPs 20 can be used for many other hybridization applications. In one example, HBPs 20 conjugated with moiety 27 and oligonucleotide 28 can be used to stain proteins on the surfaces or inside cells. FIG. 8A illustrates a protein 200 that specifically binds with an oligonucleotide 202-conjugated antibody 204. HBP 20 with an oligonucleotide 28 complementary to the sequence can then be hybridized on the oligonucleotide 202, as shown in FIG. 8B. HBP 20 can now specifically stain the protein 200 via the oligonucleotide 202-conjugated antibody 204.

It should be understood that while several examples applications for the probes are described herein, other applications are also contemplated, including but not limited to ELISA and lateral flow applications.

### Examples of High-brightness Probes With Biomolecule 28 (streptavidin)

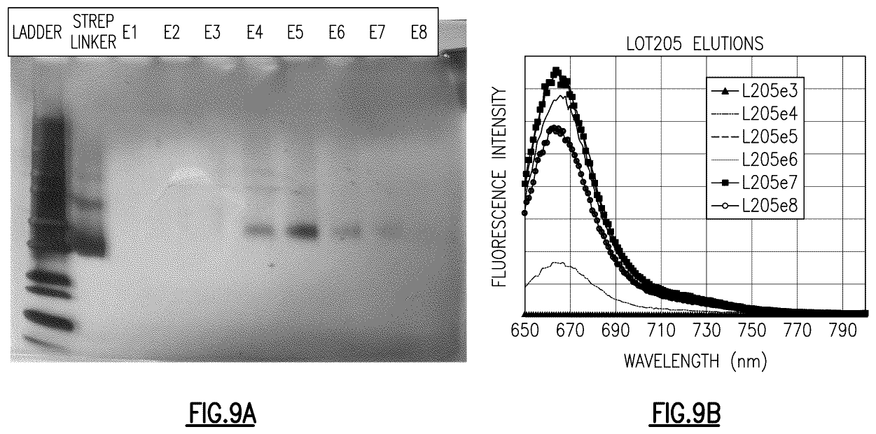

In another example of FIG. 1A, moiety 27 is a fluorescent entity, and in particular, is Cy5, which is a far-red dye. Cy5 can be conjugated to the linker 26 at R′ by any known chemistry. For instance, for the azide-amine linker 26 of FIG. 1B, a click reaction can be performed to covalently bond the R′ group of linker 26 to an DBCO or alkyne group of Cy5. The biomolecule 28 in FIG. 1A can be a protein and in particular, is a streptavidin conjugated with a linker. FIG. 9A shows the gel image after electrophoresis. As shown, HBP 20 conjugated with Cy5 and streptavidin can be purified by size exclusion column (SEC) as elutions E4, E5, E6, and E7, with the clear signal/band of streptavidin as suggested by the reference band of streptavidin-linker (Strep linker). Since the streptavidin is conjugated on the carrier 22, the corresponding band is shifted higher than the reference band, indicating successful conjugation.

FIG. 9B shows the fluorescent spectra of elutions E1 to E8. As shown, elutions E4, E5, E6, and E7 also emit fluorescence, meanings these elutions contained probes 20 conjugated with Cy5 and streptavidin. E8 is free dye-linkers without streptavidin, as shown in the gel image in FIG. 9A.

### Examples of High-Brightness Probes With Biomolecule 28 (Antibody)

In another example of FIG. 1A, moiety 27 is a fluorescent entity, and in particular, is Cy5, which is a far-red dye. Cy5 can be conjugated to the linker 26 at R′ by any known chemistry. For instance, for the azide-amine linker 26 of FIG. 1B, a click reaction can be performed to covalently bond the R′ group of linker 26 to an alkyne group of Cy5. The biomolecule 28 in FIG. 1A can be an antibody, particularly an Anti-CD 19 (Anti-CD19 linker).

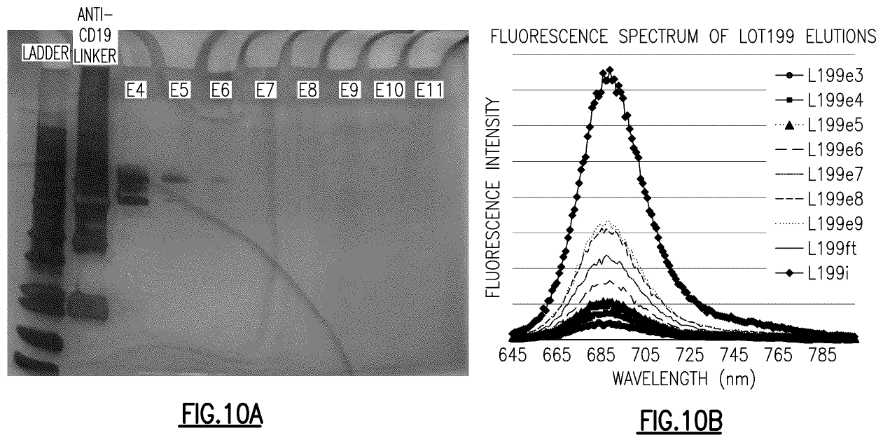

FIG. 10A shows the gel image after electrophoresis. HBP 20 conjugated with Cy5 and Anti-CD19 can be purified by size exclusion column (SEC) as elutions E4, E5, and E6, with the clear signal/band antibody as suggested by the reference band of antibody CD 19-linker (Anti-CD 19-linker). FIG. 10B shows the fluorescent spectra of the initial sample (i), the flow through (ft), and elutions E3 to E9. As shown, elutions E4, E5, and E6 emit fluorescence, meanings these elutions contained HBPs 20 conjugated with Cy5 and Anti-CD19.

In another example of HBP 20 shown in FIG. 1A, moiety 27 is a fluorescent entity, and in particular, is Pacific Blue (PB), a violet dye. PB can be conjugated to the linker 26 at R′ by any known chemistry. For instance, for the azide-amine linker 26 of FIG. 1B, a click reaction can be performed to covalently bond the R′ group of linker 26 to an alkyne group of PB. The biomolecule 28 in FIG. 1A can be an antibody, particularly an Anti-CD 19 (Anti-CD19 linker).

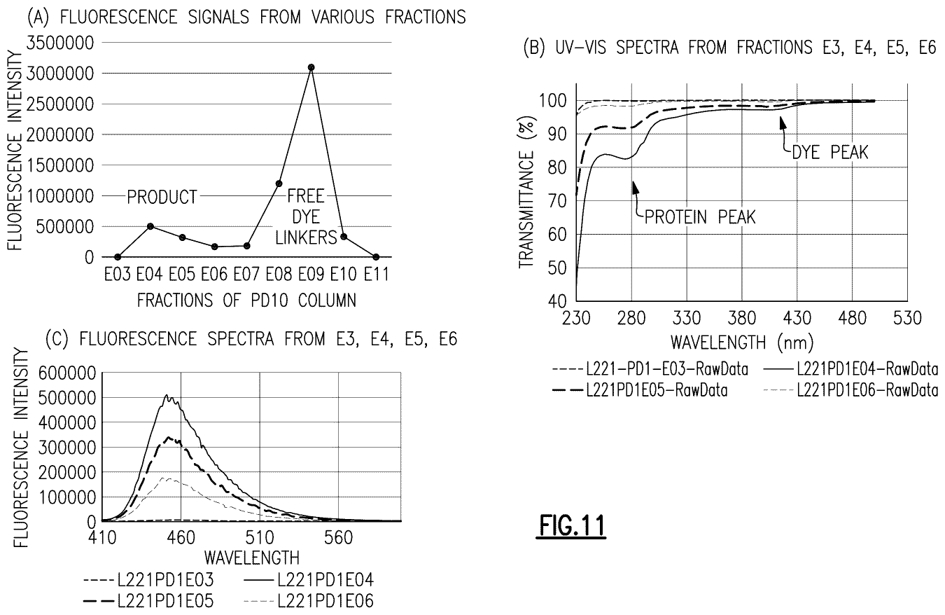

FIG. 11A shows fluorescence signals from various fractions (E3 to E11) collected from a size-exclusion column with HBP 20 (product) well separated from the unconjugated fluorescent entity (free dye-linkers). FIG. 11B shows that E4 and E5 are probes conjugated with antibodies and fluorescent entities, as evidenced by the protein absorption band (~270 nm) and dye absorption band (~405 nm). FIG. 11C supports that E4 and E5 offer strong fluorescence signals.

The preceding description is exemplary rather than limiting in nature. Variations and modifications to the disclosed examples may become apparent to those skilled in the art that do not necessarily depart from the essence of this invention. The scope of legal protection given to this invention can only be determined by studying the following claims.

What is claimed is:

1. A compound comprising:
a nanodot carrier;
at least one radical-derived moiety covalently bond with the carrier;
at least one linker having first and second functional groups, wherein the first functional group is linked to the at least one radical-derived moiety, and the second functional group is linked to at least one of a biomolecule, an oligonucleotide, and an aptamer.

2. The compound of claim 1, further comprising a second linker having first and second functional groups and a second radical-derived moiety, wherein the first functional group of the second linker is linked to the second radical-derived moiety, and the second functional of the second linker group is linked to a fluorescent entity.

3. The compound of claim 2, wherein the nanodot carrier has at least one polar group, and wherein the first and second radical-derived moieties are each covalently bonded with the carrier at one of the at least one polar groups.

4. The compound of claim 3, wherein the at least one polar group is a hydroxyl (—H) group.

5. The compound of claim 1, wherein the nanodot carrier is an h-BN nanodot carrier.

6. The compound of claim 5, wherein the nanodot carrier has dimensions between about 1 and about 100 nm.

7. The compound of claim 6, wherein the nanodot carrier has dimensions between about 1 and about 20 nm.

8. The compound of claim 5, wherein the nanodot carrier comprises less than 30 layers of h-BN.

9. The compound of claim 8, wherein the nanodot carrier comprises between about 1 and 10 layers of h-BN.

10. The compound of claim 1, wherein at least ten radical-derived moieties are covalently bonded with the carrier, each radical-derived moieties is linked to a linker, and each linker is linked to at least one of a moiety, a biomolecule, an aptamer, or an oligonucleotide.

11. The compound of claim 10, wherein the moiety is a chelating agent.

12. A method of making a probe, comprising:
mechanically processing nanodots in polar liquid to create imperfections on the nanodots;
treating the nanodots to provide polar groups at the imperfections;
associating radicals with the polar groups at the imperfections; and
linking a linker with the nanodots via one of the associated radicals at a first functional group of the linker.

13. The method of claim 12, further comprising linking a fluorescent entity to a second functional group of the linker.

14. The method of claim 12, wherein the mechanically processing includes agitation.

15. The method of claim 14, wherein the agitation is accomplished by sonication or by homogenizer.

16. The method of claim 12, wherein the treating is an acid treatment, and wherein the polar groups are hydroxyl (—OH) groups.

17. The method of claim 12, further comprising using the probe for fluorescence in-situ hybridization (FISH) to detect or quantify at least one of RNAs, DNAs, genes, and proteins via oligonucleotide-conjugated antibodies in fixed or live cell samples or fixed or live tissue samples.

18. The method of claim 17, wherein the probe is configured to be hybridized on the oligonucleotide-conjugated antibodies.

19. The method of claim 12, further comprising using the probe in spatial omics to image, localize or map at least one of RNAs, DNAs, genes, and proteins via oligonucleotide-conjugated antibodies in fixed or live cell samples or fixed or live tissue samples.

20. The method of claim 19, wherein the probe is configured to be hybridized on the oligonucleotide-conjugated antibodies.

21. The method of claim 12, wherein the linker is a first linker, and further comprising:
linking a second linker with the nanodots via a radical-derived moieties of the associated radicals at a first functional group of the second linker;
linking a fluorescent entity to a second functional group of the first linker; and
linking an oligonucleotide, an apatamer, or a biomolecule to a second functional group of the second linker.

22. The method of claim 21, wherein an oligonucleotide is linked to the second functional group of the second linker, and further comprising using the probe in a sensor to detect proteins or biomarkers extracted from body fluids, wherein the oligonucleotide is configured to bind with the proteins or biomarkers.

23. The method of claim 21, wherein the biomolecule is streptavidin and further comprising using the probe to detect biotinylated molecules.

24. The method of claim 21, wherein the biomolecule is an antibody, and further comprising using the probe to detect proteins inside a cell, outside cells, or on a cell surface.

25. The method of claim 21 further comprising using the probe to detect proteins on extracellular vesicles (EV) and exosomes.
